(19) United States
(12) Patent Application Publication
(10) Pub. No.: US 2023/0240847 A1
(43) Pub. Date: Aug. 3, 2023

(54) Heart Valve Commissure Bridge for Valve Repair

(71) Applicant: St. Jude Medical, Cardiology Division, Inc., St. Paul, MN (US)

(72) Inventors: William H. Peckels, Robbinsdale, MN (US); Heath Marnach, Minneapolis, MN (US); Michael J. Urick, Chaska, MN (US)

(73) Assignee: St. Jude Medical, Cardiology Division, Inc., St. Paul, MN (US)

(21) Appl. No.: 18/055,533

(22) Filed: Nov. 15, 2022

Related U.S. Application Data
(60) Provisional application No. 63/306,136, filed on Feb. 3, 2022.

(51) Int. Cl. A61F 2/24

(52) U.S. Cl. CPC A61F 2/2445; A61F 2/2466; A61F 2210/0014; A61F 2220/0075

(57) ABSTRACT

A medical device for treating a native heart valve may include a frame formed of shape-memory material, the frame including a central bar extending between a first leg and a second leg of the frame. The frame may be transitionable between a delivery condition and a deployed condition. The first leg and the second leg may each having a curved portion with a concave outer surface in the deployed condition. The concave outer surface may be sized and shaped to engage a corresponding native commissure of the native heart valve so that, when the frame is deployed within the native heart valve, the central bar bridges across the native heart valve.

### CROSS-REFERENCE TO RELATED APPLICATIONS

This application claims priority to the filing date of U.S. Provisional Patent Application No. 63/306,136, filed Feb. 3, 2022, the disclosure of which is hereby incorporated by reference herein.

### BACKGROUND OF THE DISCLOSURE

The human heart includes four valves that help to control the directionality of blood flow, allowing for blood to flow in an antegrade direction and preventing blood from flowing in a retrograde direction. These valves include (i) the left atrioventricular valve (also referred to as the mitral valve); (ii) the right atrioventricular valve (also referred to as the tricuspid valve); (iii) the aortic valve; and (iv) the pulmonary valve. The atrioventricular valves separate the corresponding atrium from the corresponding ventricle. The aortic valve separates the left ventricle from the aorta. The pulmonary valve separates the right ventricle from the pulmonary artery.

Each of these valves is formed of one or more leaflets, and each leaflet is attached to an adjacent leaflet at a valve commissure. Over time, one or more leaflets and/or the native valve annulus may become diseased or otherwise damaged. For example, the leaflets may become calcified and/or their shapes may be distorted, which may impact the ability of the leaflets to sufficiently close (also referred to as coapting). If the leaflets of a native heart valve cannot close properly, blood may leak in the retrograde direction. There are many available treatments to correct the functioning of a malfunctioning heart valve. For example, surgical heart valves or transcatheter heart valves may be implanted to provide an entirely new prosthetic heart valve that takes over functioning of the native heart valve. Other treatments have been used specifically for the mitral and tricuspid valves, such as leaflet clips that clip the leaflets together in an attempt to provide a better seal.

Despite the availability of various treatments to replace or repair the functioning of a native heart valve, it would still be desirable to have additional options, including minimally invasive procedures that are effective in mitigating regurgitation across a native heart valve.

### BRIEF SUMMARY OF THE DISCLOSURE

According to one aspect of the disclosure, a medical device for treating a native heart valve may include a frame formed of shape-memory material, the frame including a central bar extending between a first leg and a second leg of the frame. The frame may be transitionable between a delivery condition and a deployed condition, and the frame may be formed of a solid or hollow tube. The first leg and the second leg may each having a curved portion with a concave outer surface in the deployed condition. The concave outer surface may be sized and shaped to engage a corresponding native commissure of the native heart valve so that, when the frame is deployed within the native heart valve, the central bar bridges across the native heart valve.

According to another embodiment of the disclosure, a method of treating a mitral valve of a patient includes loading a medical device into a catheter in a delivery condition. The catheter may be advanced through a vasculature of the patient to a right atrium of the patient, through an atrial septum, and into a left atrium of the patient while the catheter maintains the medical device in the delivery condition. The medical device may be deployed from the catheter into engagement with the mitral valve, the medical device transitioning from the delivery condition to a deployed condition during the deployment. When the medical device is in the deployed condition, a first leg of the medical device engages a first commissure of the mitral valve, a second leg of the medical device engages a second commissure of the mitral valve, and a central bar extends from the first leg to the second leg so that the medical device presses outwardly on the first commissure and the second commissure.

### BRIEF DESCRIPTION OF THE DRAWINGS

FIG. 1 is a highly schematic cutaway representation of a human heart showing various delivery approaches.

FIG. 2 is a highly schematic representation of a native mitral valve and associated cardiac structures.

FIG. 3A is a highly schematic illustration of a mitral valve treatment device, according to one embodiment of the disclosure, positioned within a native mitral valve annulus.

FIG. 3B is a perspective view of the mitral valve treatment device of FIG. 3A in a relaxed or deployed condition.

FIG. 3C is a perspective view of a mitral valve treatment device similar to that of FIG. 3A in a collapsed or delivery condition.

FIG. 3D is an enlarged view of dashed circle 3D of FIG. 3B.

FIG. 3E is an enlarged view of dashed circle 3E of FIG. 3C.

FIG. 3F is an enlarged view of an alternate embodiment of the recesses and cut-outs shown in FIG. 3E.

FIG. 4A is a highly schematic illustration of a mitral valve treatment device, according to another embodiment of the disclosure, positioned within a native mitral valve annulus.

FIG. 4B illustrates a portion of the treatment device of FIG. 4A in a delivery condition.

FIG. 5A is a highly schematic illustration of a mitral valve treatment device, according to a further embodiment of the disclosure, positioned within a native mitral valve annulus and viewed from an atrial side of the valve.

FIG. 5B illustrates the same device as FIG. 5A, but viewed from a ventricular side of the valve.

FIG. 5C is a highly schematic cut-away view of the treatment device of FIGS. 5A-B deployed within the native mitral valve.

FIG. 5D is a highly schematic illustration of the mitral valve treatment device of FIG. 5A positioned within the native mitral valve annulus with an additional support member.

FIG. 5E is a highly schematic illustration of the mitral valve treatment device of FIG. 5A positioned within the native mitral valve annulus with an alternate additional support member.

FIG. 6A is a side view of a heart valve treatment device according to another aspect of the disclosure.

FIG. 6B is a highly schematic illustration of the treatment device of FIG. 6A in a deployed or expanded condition within a native mitral valve.

FIG. 6C is a perspective view of the treatment device of FIG. 6A in an expanded or deployed condition.

FIG. 7 in a schematic illustration of the treatment device of FIG. 3A being deployed from a catheter that integrates a tensioning mechanism.

FIG. 8 is a perspective view of a treatment device for use in tri-leaflet valves according to an aspect of the disclosure.

FIG. 9 is a highly schematic illustration of a treatment device for use in bi-leaflet valves according to another aspect of the disclosure.

FIG. 10A is a highly schematic illustration of a central bar of a treatment device in a first length condition.

FIG. 10B is the central bar of FIG. 10B in a second length condition longer than the first length condition.

### DETAILED DESCRIPTION

As used herein, the term “proximal,” when used in connection with a valve leaflet repair device, generally refers to the end of the device that is closest to a user of the device (e.g. a surgeon or other hospital personnel), while the term “distal” refers to the opposite end. In other words, the leading end of the device may be positioned distal to the trailing end of the device. Also, as used herein, the terms “substantially,” “generally,” and “about” are intended to mean that slight deviations from absolute are included within the scope of the term so modified. Generally, materials described as being suitable for components in one embodiment may also be suitable for similar or identical components described in other embodiments.

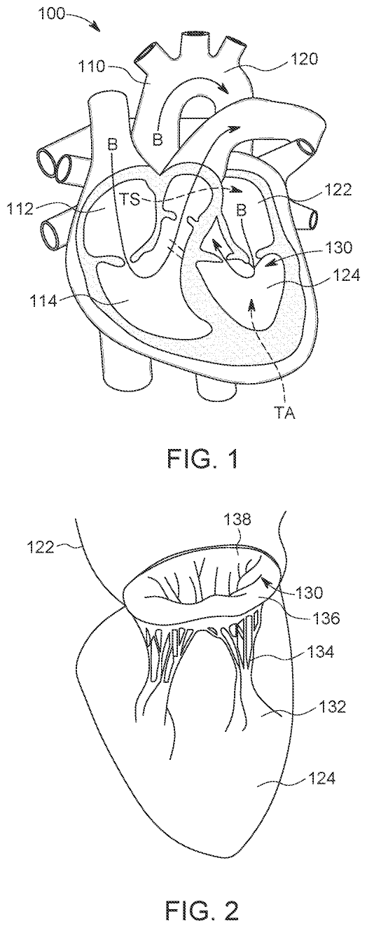

FIG. 1 is a highly schematic cutaway representation of human heart 100. The human heart includes two atria and two ventricles: right atrium 112 and left atrium 122, and right ventricle 114 and left ventricle 124. Heart 100 further includes aorta 110 and aortic arch 120. Disposed between left atrium 122 and left ventricle 124 is mitral valve 130. Mitral valve 130, also known as the bicuspid valve or left atrioventricular valve, is a dual-flap valve that opens as a result of increased pressure in left atrium 122 as it fills with blood. As atrial pressure increases above that of left ventricle 124, mitral valve 130 opens and blood passes into left ventricle 124. Blood flows through heart 100 in the direction shown by arrows “B”.

A dashed arrow, labeled “TA,” indicates a transapical approach of implanting a device, such as a prosthetic heart valve or a valve and/or leaflet repair device, in this case to treat the mitral valve. In transapical delivery, a small incision is made between the ribs and into the apex of left ventricle 124 to deliver the treatment device to the target site. A second dashed arrow, labeled “TS,” indicates a transseptal approach of implanting a treatment device in which the device is passed through the septum between right atrium 112 and left atrium 122. Typically, in the transseptal approach, access to the left atrium is gained by advancing a catheter through the femoral vein and then the inferior vena cava, at which point a needle or other object is used to pierce the atrial septum to provide access to the mitral valve 130. However, in the transseptal approach, access to the left atrium may be gained from the superior vena cava, for example via the jugular vein. It should be understood that similar approaches may be used to reach the tricuspid valve, although it should be understood that piercing the atrial septum would not be necessary when treating the tricuspid valve using an approach from the inferior or superior vena cava. Other approaches for implanting a heart valve treatment device are also possible other than those explicitly described above.

FIG. 2 is a more detailed schematic representation of native mitral valve 130 and its associated structures. As previously noted, mitral valve 130 includes two flaps or leaflets, posterior leaflet 136 and anterior leaflet 138, disposed between left atrium 122 and left ventricle 124. Cord-like tendons, known as chordae tendineae 134, connect the two leaflets 136, 138 to the medial and lateral papillary muscles 132. During atrial systole, blood flows from higher pressure in left atrium 122 to lower pressure in left ventricle 124. When left ventricle 124 contracts in ventricular systole, the increased blood pressure in the chamber pushes leaflets 136, 138 to close, preventing the backflow of blood into left atrium 122. Since the blood pressure in left atrium 122 is much lower than that in left ventricle 124, leaflets 136, 138 attempt to evert to the low pressure regions. Chordae tendineae 134 prevent the eversion by becoming tense, thus pulling on leaflets 136, 138 and holding them in the closed position. Although not shown separately, the tricuspid valve may have a generally similar shape to the mitral valve, although the mitral valve typically has a more complex shape. The tricuspid valve, as its name implies, typically has three leaflets, as opposed to the typical two leaflets found in the mitral valve

Two types of treatments of native heart valves were described above as prosthetic heart valves that mostly or fully replace the functionality of the native heart valve, as well as leaflet clips that clip native leaflets together in an attempt to enhance sealing between the remaining, unclipped portions of the native leaflets. The heart valve treatment devices described herein may be used to help reduce or eliminate regurgitation across the native heart valve by stretching the native commissures of the native valve leaflets apart, which may result in better leaflet coaptation and, thus, reduce or eliminate regurgitation.

It should be understood that, although the heart valve treatment devices are generally described below in connection with treating the mitral valve, the devices could be used, with or without modification, to treat other native valves of the heart, such as the tricuspid valve, or the aortic or pulmonary valve.

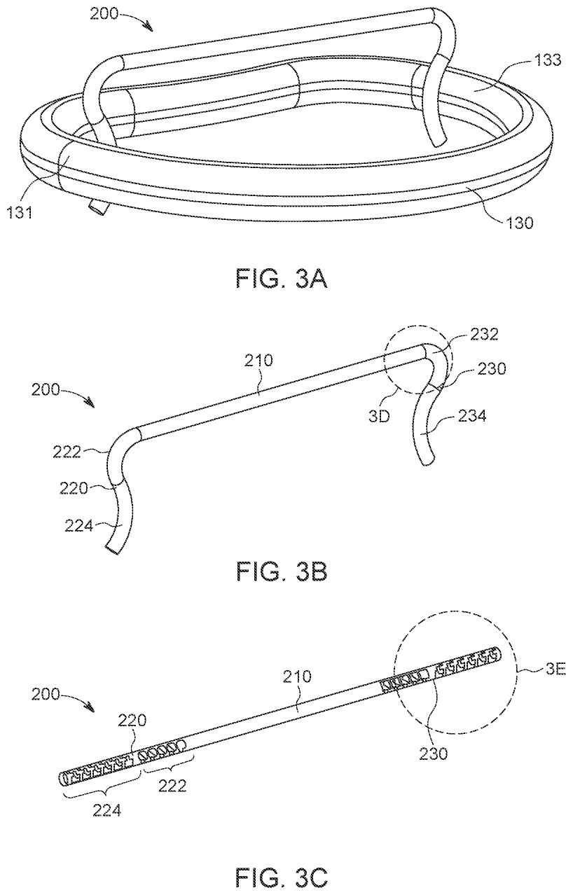

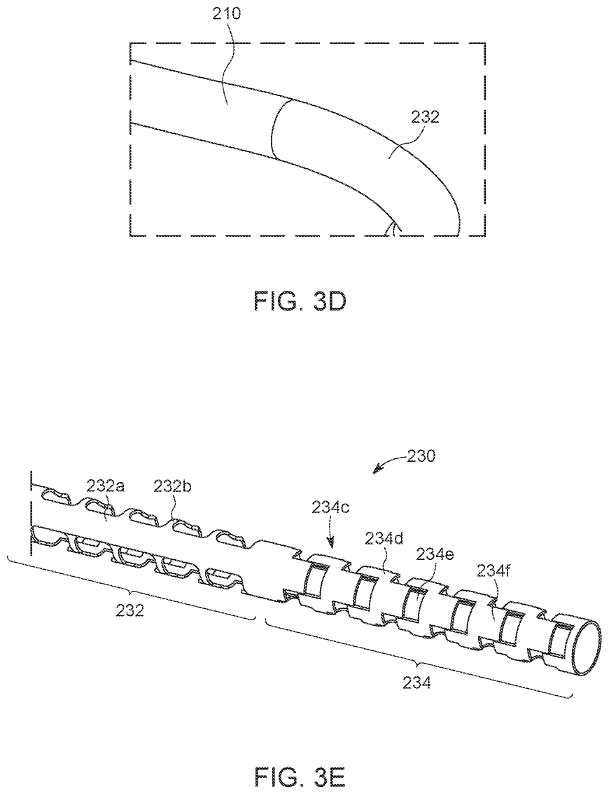

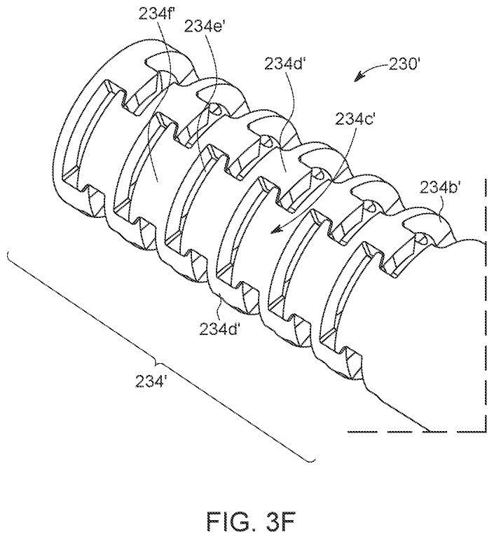

FIG. 3A is a perspective view of a heart valve treatment device 200 deployed within the annulus of a native mitral valve 130. It should be understood that FIG. 3A is a simplified drawing, omitting structures of the native mitral valve 130 include leaflets, chordae tendineae, etc. — many of which can be seen in FIG. 2. In the view of FIG. 3A, the top of the page is the inflow side (or the atrial side) while the bottom of the page is the outflow side (or ventricular side). As shown in FIGS. 2-3A, the native mitral valve 130 may be generally “D”-shaped or oval and may include two commissures 131, 133 where the posterior leaflet 136 and the anterior leaflet 138 meet each other.

Referring now to FIGS. 3A-3C, the heart valve treatment device 200 may be formed as a tube of material having shape memory properties. One exemplary shape memory material is nickel titanium alloy, such Nitinol, although it should be understood that other shape memory materials may be suitable in place of (or in addition to) Nitinol. Treatment device 200 may include three main portions, including a central bar 210, first leg 220, and second leg 230. Generally, in use, the treatment device 200 is deployed into the annulus of the native mitral valve 130 so that the first leg 220 and second leg 230 are nested within the native valve commissures 131, 133, such that the treatment device bridges the commissures 131, 133 and puts outward pressure on the commissures. This outward force on the native commissures 131, 133 may help to tension the native valve leaflets 136, 138, resulting in better coaptation of the native leaflets, minimizing or eliminating any regurgitation across the native mitral valve 130. The central bar 210 is preferably oriented on the inflow or atrial side of the native mitral valve 130 when deployed, for example to avoid interfering with the native leaflets 136, 138 which extend in the outflow direction (or ventricular direction) from the annulus of the native mitral valve 130. Although treatment device 200 is described as being formed from a tube of shape-memory material, other configurations may be appropriate. For example, in some embodiments, the treatment device 200 may be formed of braided strands of a shape-memory material, including braiding into a generally cylindrical or tubular shape. In other embodiments, the treatment device 200 may have a cross-sectional profile other than circular, including oval, square, rectangular, etc.

Structurally, treatment device 200 may be formed as a cylindrical tube (e.g. with a circular cross-section), and then shape-set (for example via heat setting) to take the desired shape, such as that shown in FIGS. 3A-B. The set shape of the treatment device 200 may include a generally straight portion, identified as central bar 210. Extending from either side of the central bar may be two legs 220, 230, which are preferably mirror images to each other, although in some embodiments the two legs 220, 230 may not have identical mirrored shapes. First leg 220 may include a first curved portion 222 extending from a first end of central bar 210, and a second curved portion 224 extending from the first curved portion 222 and extending to a first terminal end of the treatment device 200. Similarly, second leg 230 may include a first curved portion 232 extending from a second end of central bar 210, and a second curved portion 234 extending from the first curved portion 232 and extending to a second terminal end of the treatment device 200.

The first curved portions 222, 232 may each be curved so that the outer surfaces of the first curved portions are convex and the inner surfaces of the first curved portions are concave, such that the first curved portions has a curvature that generally bends in a direction back toward the central bar 210. The transition between the first curved portions 222, 232 and their respective second curved portions 232, 234 may be points of inflection, for example so that second curved portions each generally bend in a direction, from the inflection point, away from the central bar 210. Using terminology similar to the description of the first curved portions 222, 232, the second curved portions 224, 234 may have an outer surface that has a concave curvature, and an inner surface that has a convex curvature. In the description in this paragraph, it should be understood that “inner” and “outer” are terms relative to a center of the treatment device 200, as opposed to referring to the interior and exterior of the tube that forms the treatment device 200. In other words, in the context of this paragraph, an “inner” surface is one that generally faces toward a center of the treatment device 200, whereas an “outer” surface is one that generally faces away from the center of the treatment device 200. With the configuration described above, the outer surfaces of the second curved portions 224, 234 form a concave curvature in which the commissures 131, 133 of the native mitral valve 130 may nest. As will become apparent from the description below, the treatment device 200 is preferably shape set so that, when no force is applied to the treatment device 200, the distance between the outer surfaces of the second curved portions 224, 234 is greater than the distance between the commissures 131, 133 of the annulus of the native heart valve 130. With this configuration, when the treatment device 200 is deployed within a heart valve such as native mitral valve 130, the treatment device 200 “wants” to extend to its set-shape, but is unable to do so completely because the commissures 131, 133 are spaced together more closely than the legs 220, 230 would be if the treatment device 200 were able to fully return to its set shape. Stated differently, when the treatment device 200 is deployed in the native mitral valve 130, the legs 220, 230 (and specifically the outer surfaces of the second curved portions 224, 234) press against the commissures 131, 133, causing the native valve leaflets 136, 138 to be slightly tensioned while also maintaining the position of the treatment device 200 within the native mitral valve 130.

Although treatment device 200 is preferably shape-set to the shape shown in FIGS. 3A-B, or a shape similar thereto, the treatment device 200 is preferably transitionable to a collapsed or delivery condition, similar to that shown in FIG. 3C. Although the term “collapsed condition” is used herein, it should be understood that, upon transitioning from the condition shown in FIG. 3B to that shown in FIG. 3C (or vice versa), there need not be any true collapsing (or expanding) of the treatment device. Rather, when in the collapsed or delivery condition shown in FIG. 3C, the treatment device is effectively in a cylindrical shape so that is has small profile to allow for delivery into the body within a catheter having a small diameter. For example, a catheter as small as between about 10 French (3.33 mm) and 24 French (8 mm) outer diameter may be used to transseptally deliver the treatment device 200, the catheter maintaining the treatment device in the collapsed or delivery condition shown in FIG. 3C until the treatment device 200 is deployed from the catheter, allowing the treatment device 200 to return (or attempt to return) to its set shape, similar to that shown in FIGS. 3A-B. In other words, the delivery catheter used to delivery treatment device 200 may be smaller than a typical delivery catheter used in known prosthetic heart valves or valve/leaflet repair devices, such as leaflet clips.

Treatment device 200 may be provided as a solid or hollow tube, and then shape-set into the desired deployment shape. In some iterations, the treatment device 200 may be laser-cut or otherwise modified to help the treatment device 200 maintain or achieve certain shape profiles. FIG. 3D illustrates an enlarged view of the first curved portion 232 of second leg 230, focusing on the dashed circle 3D of FIG. 3B. Similarly, FIG. 3E illustrates an enlarged view of the second leg 230, focusing on the dashed circle 3E of FIG. 3C. FIGS. 3C and 3E illustrate the treatment device 200 after having been laser cut (or otherwise modified) to include segmented cut-outs in the legs 220, 230 to assist with the transition between the shapes shown in FIGS. 3B and 3C. However, it should be understood that the segmented cut-outs may be entirely omitted, or provided in other formats to provide similar functions.

Focusing on FIG. 3E, the second leg 230 is illustrated in the collapsed or delivery condition. The first curved portion 232 and the second curved portion may each include cut-outs or recesses, which may be formed by any suitable mechanism including laser cutting, with the recesses or cut-outs facing in opposite directions. As should be clear, in this embodiment the second leg 230 is provided as a hollow tube. The first curved portion 232 may include a substantially continuous spine 232a running along the length of the second leg 230 between the central bar 210 and the transition point (or inflection point) to the second curved portion 234. The spine 232a may be a wall of the tube that forms the treatment device 200, and the spine 232a, when in the deployed condition, may be on the inner surface of the first curved portion 232. The second curved portion 234 also includes a spine, but that spine is preferably on the opposite side (e.g. diametrically opposed to spine 232a), so that in the deployed condition, the spine of the second curved portion 234 is on the outer surface of the second curved portion 234 and is configured to contact and press against one of the native commissures 131, 133.

Still referring to FIG. 3E, the spine may include two ribs 232b or thin lateral projections extending generally orthogonally to the spine 232a. The ribs 232b may also form part of the wall of the tube that forms the treatment device 200. A relatively large empty space may be formed between adjacent pairs of ribs 232b along the length of the spine 232a. The spine 232a and ribs 232b are shown and described in connection with first curved portion 232 because they are most easily seen in FIG. 3E on the first curved portion 232. However, it should be understood that the same components may be provided on the second curved portion 234b, but they are not easily visible in the view of FIG. 3E. The other components of the segmented cut-outs are described in connection with second curved portion 234, as they are most easily visible in FIG. 3E in connection with the second curved portion 234.

Each of the ribs in a pair of ribs may transition to a segment 234c positioned generally opposite the spine. Each segment 234c may include two side portions 234d that define an open space 234e circumferentially therebetween. The two side portions 234d may join at a projection 234f, the projection 234f generally positioned in or adjacent to the open space 234e of an adjacent segment 234c. However, the projection 234f of one segment 234c is preferably not directly coupled to an adjacent segment 234c. With this repeating configuration, as the spine bends or curves, the projections of segments may nest within the open space provided by adjacent segments, which may help the treatment device 200 more easily contour into the desired curved shape. It should be understood that other types of segmentations may be provided to assist with helping to form the curvature of the treatment device 200, such as scores or other shaped cut-outs.

The cut-outs or recesses may provide additional and/or alternate functionality to that described above. For example, upon transitioning to the deployed shape, the segments could “lock out” to help maintain the curved portions 222, 224, 232, 234 in their desired shapes. For example, the contact between structures of adjacent segments may provide frictional forces to help prevent the curved portions from changing shape due to, for example, typical forces that may be experienced during normal operation from movement of the heart muscle, blood flow, etc. Still further, the cut-outs or recesses may assist with maintaining the shape of the delivery catheter during delivery. For example, if the treatment device 200 is formed of a solid tube of nitinol (or other shape-memory material) and shape-set to the desired curvature shown in the figures, the tendency of the treatment device 200 to attempt to take its set shape may, to some degree, “overpower” the catheter which is trying to maintain the treatment device 200 in a straight or delivery configuration. Thus, instead of the delivery catheter maintaining the treatment device 200 in a full straight delivery condition, the treatment device 200 instead may start to return, to some degree, to the set shape and in the process cause the delivery catheter to bend. The likelihood of this occurring, however, may be reduced or eliminated by introducing the recesses or cut-outs described above. In fact, the exact shape and positioning of the recesses or cut-outs may be fine-tuned for optimization between the strength of the catheter and the strength of the treatment device 200. In other words, the cut-outs or recesses may be designed to maximize the strength of the treatment device 200 while it is in its deployed shape in the native heart valve, while not being so strong as to cause the delivery catheter to deform during delivery of the treatment device 200. This fine tuning may be performed, at least in part, by increasing or decreasing the amount of material forming a continuous solid wall, in the treatment device 200, particularly at the areas where the treatment device will bend out of the straight delivery shape upon deployment into the patient (e.g. portions 222, 224, 232, 234). Still further, structure forming the recesses or cut-outs may provide additional surfaces that may be able to grip or otherwise engage the tissue upon deployment, helping to act as anchors.

For example, some of the surfaces adjacent the cut-outs or recesses may, upon deployment, dig into tissue and act as a tine, hook, or barb, helping to maintain the treatment device 200 in the desired position relative to the native valve annulus.

As should be understood from the above, although one particular configuration of recesses and cut-outs is illustrated in FIG. 3E, various other configurations may be suitable. One additional exemplary configuration is illustrated in FIG. 3F. In FIG. 3F, a portion of a second leg 230′ is illustrated in the collapsed or delivery condition. A first curved portion (not illustrated in FIG. 3F) and a second curved portion 234′ may each include cut-outs or recesses, which may be formed by any suitable mechanism including laser cutting, with the recesses or cut-outs facing in opposite directions in each of the curved portions, although only one curved portion is shown in FIG. 3F. As should be clear, in this embodiment the second leg 230′ is provided as a hollow tube. The second curved portion 234′ may include a substantially continuous spine (not visible in the view of FIG. 3F) running along the length of the second leg 230′, substantially similar to the spins shown and describe in connection with FIG. 3E. The spine may include two ribs 234b′ or thin lateral projections extending generally orthogonally to the spine. The ribs 234b′ may also form part of the wall of the tube that forms the treatment device. Each of the ribs in a pair of ribs may transition to a segment 234c′ positioned generally opposite the spine. Each segment 234c′ may include two side portions 234d′ that define an open space 234e′ circumferentially therebetween. The two side portions 234d′ may join at a projection 234f. Each projection 234f′ may include two lateral tabs that fit within a pair of shoulders, one shoulder of each pair formed by one side portion 234d′ or each pair of side portions 234d′. The configuration shown in FIG. 3F may have generally similar functionality as that recesses and cut-outs described in connection with FIG. 3E. However, the tabs and shoulders may provide for additional control of the contouring of the treatment device upon deployment and once deployed. For example, the tabs and shoulders may help ensure that the second curved portion 234′ bends only in the desired directions during deployment.

In a typical use of treatment device 200, it may first be formed as described above. For example, a hollow tube of Nitinol may first be laser cut to have the segmented cut-outs described above, and then heat set to a shape similar to that shown in FIG. 3A. However, it should be understood that in other embodiments the tube may be solid, in which case the segmented cutouts may be omitted. Preferably, a plurality of treatment devices 200 of different sizes are provided, and the physician chooses the most suitable size treatment device 200 based on the patient's mitral valve anatomy. For example, as noted above, the physician may choose a treatment device 200 having a size in which the length between the concave outer surfaces of the second curved portions 224, 234 of the legs 220, 230 is larger than the distance between the native commissures 131, 133, to help ensure that upon deployment of the treatment device 200, the legs 220, 230 press against the commissures 131, 133 with an outward force. Once the treatment device 200 of the desired size is chosen, it may be loaded into a catheter, with the treatment device 200 transitioning to the straight delivery or collapsed condition during the loading. If the legs 220, 230 are mirror images of one another, it may not matter whether leg 220 is the leading end of the treatment device 200, or if leg 230 is the leading end of the treatment device. Once the treatment device 200 is in the delivery condition within the delivery catheter, the delivery catheter may be inserted into the patient's vasculature, for example into the femoral vein, and advanced until it reaches the right atrium. If not already done, a needle may be used to pierce the atrial septum. If the treatment device 200 is hollow, the needle may be passed through a center of the treatment device 200. However, in other embodiments, a needle may first be used to pierce the atrial septum, and a guidewire may be advanced through the atrial septum, and the guidewire may then be used as a rail for the delivery catheter housing the treatment device 200. It should be understood that these steps are merely exemplary, and other specific techniques may be used to advance the delivery catheter housing the treatment device 200 into the left atrium.

Once the delivery device is within the left atrium, the distal tip of the delivery device may be positioned near or adjacent one of the native commissures, and the treatment device 200 may be deployed from the delivery catheter. In one example, a push rod or other device may be advanced within the delivery catheter to push the treatment device 200 out of an open distal end of the delivery catheter. In another embodiment, a distal sheath of the delivery catheter may be withdrawn proximally, and the withdrawal of the distal sheath exposes the treatment device 200. In either case, as the treatment device exits the constrains of the delivery catheter, the shape memory properties of the treatment device 200 result in the treatment device 200 beginning to take its set shape. For example, one of the legs will be deployed first, and as the second curved portion of that leg is deployed, it is positioned to contact the corresponding native commissures. As deployment continues, the delivery catheter may be withdrawn and/or maneuvered near the opposing commissure, so that as the trailing leg of the treatment device 200 deploys, the second curved portion of that leg will hook around or nest with the remaining native commissure. After the treatment device 200 is fully deployed, it may have the configuration shown in FIG. 3A, with the two legs 220, 230 of the treatment device 200 stretching the native commissure 131, 133, putting tension on the native leaflets 136, 138 and enhancing leaflet cooptation.

It should be understood that treatment device 200 may not need any special anchoring features beyond the force resulting from the shape memory properties of the treatment device 200. For example, the forces from blood flowing through the native mitral valve 130 may be small relative to the anchoring force provided by the shape memory properties of the treatment device 200. Still further, the concave shape of the second rounded portions of the legs 220, 230 may provide a high amount of stability of the position of the treatment device 200 relative to the native mitral valve 130. However, in other embodiments, particularly those described below which include additional structure on the treatment device, anchoring features may be provided. For example, barbs, tines, hooks, or other features may be provided on the portions of the legs 220, 230 that will contact the commissures. These features may function to pierce tissue to provide robust anchoring, or simply to provide high friction without actually piercing through tissue.

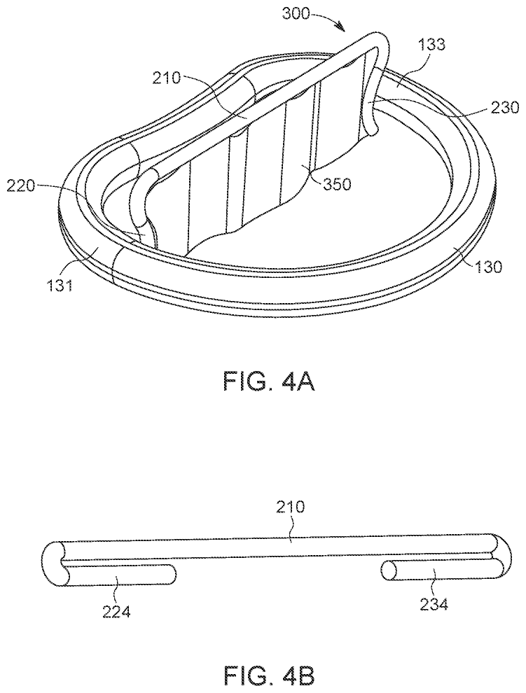

FIG. 4A illustrates a valve treatment device 300 according to another aspect of the disclosure. Treatment device 300 may, effectively, be the treatment device 200 described above, with a sheet member 350 added to treatment device 200. The sheet member 350 may provide a surface against which native valve leaflets 136, 138 may press when the native valve leaflets coapt. As described in greater detail below, the sheet member 350 may help fill any gaps that would otherwise be present between the coapted native valve leaflets 136, 138 in the absence of the sheet member 350.

Referring still to FIG. 4A, the valve treatment device 300 may include a frame that is similar or identical to treatment device 200, and is thus not described in great detail again here. However, it should be understood that treatment device may include central bar 210, first leg 220, and second leg 230, and those components may be structurally similar or identical to those described above in connection with treatment device 200, including all alternatives described. For example, the frame of treatment device 300 is preferably formed of a hollow or solid tube of shape memory material, such as a nickel titanium alloy, and heat or otherwise shape set to the shape shown in FIG. 4A. The main structural difference between treatment devices 200 and 300 is that treatment device 300 includes a sheet member 350 coupled to the frame. Sheet member 350 may be a single layer (although in some circumstances it may be multi-layered) sheet of material that is biocompatible. For example, the sheet member 350 may be formed of tissue, such as porcine or bovine tissue, including porcine or bovine pericardial tissue. In other embodiments, the sheet member 350 may be formed of a synthetic material (such as a polymer fabric), such as PET, PTFE, any other suitable biocompatible sealing material, and/or combinations thereof Sheet member 350 may have a shape that generally follows the contours of the frame when the frame is in the expanded or deployed condition shown in FIG. 4A. For example, the top of sheet member 350 may generally follow the contours of the central bar 210, and the sides of the sheet member 350 may generally follow the contours of the first leg 220 and the second leg 230. The bottom of the sheet member 350 may be generally straight or contoured as desired.

When the treatment device 300 is deployed into a native heart valve, such as native mitral valve 130, the treatment device 300 is held in place in substantially the same manner as described above in connection with treatment device 200. Treatment device 300 may also press against the native commissures 131, 133 to tension the native leaflets 136, 138 in substantially the same manner as described above in connection with treatment device 200. When in the deployed condition within a native valve annulus, the sheet member 350 preferably extends far enough in the outflow direction of the native valve annulus so that when the native valve leaflets 136, 138 coapt with each other (not shown in FIG. 4A), the native leaflets 136, 138 press against opposite sides of the sheet member 350. The presence of the sheet material 350 between the native valve leaflets 136, 138 when the native valve leaflets are coapted may fill any gap between the native leaflets that would otherwise exist in the absence of the sheet material 350. In other words, treatment device 300 may mitigate regurgitation in the same manner described above in connection with treatment device 200, but may alternatively or additionally mitigate regurgitation by sheet member 350 filling any gaps between the native leaflets 136, 138 when the native leaflets coapt. To be clear, although it is preferred that treatment device 300 provide native leaflet tension in the same manner as described above in connection with treatment device 200, treatment device 300 may alternatively provide less tension to the native leaflets and its primary mode of mitigating regurgitation may be via sheet member 350. The embodiment shown in FIG. 4A may be particularly useful at treating leaflet prolapses or flails. In some embodiments, the sheet member 350 may be sized so that, upon implantation of the treatment device 300, the sheet member 350 extends a slight distance into the patient's ventricle to allow the native leaflets coapt early (e.g. before the mitral plane). The sheet member 350 may interrupt the arc of the native leaflets, shortening the movement but also allowing the native leaflet to fold over onto treatment device 300.

Sheet member 350 may be fixed to the frame of treatment device 300 via any suitable fashion. For example, sheet member 350 may be sutured to the central bar 210 and/or the first leg 220 and/or the second leg 230 in any desirable pattern. In some embodiments, the frame may include apertures, notches, or other features to assist with the suturing process. However, the sheet member 350 may be fixed to the frame of treatment device 300 in other manners, including via biocompatible adhesives, etc. In some embodiments, the sheet member 350 may be folded over central bar 210, with the free ends of the sheet member 350 being sutured or otherwise fixed together. If the sheet member 350 is formed of fabric, it may be ultrasonically welded to fix it to the treatment device 300. However, as noted above, these are merely exemplary options for attachment. In other embodiments, one or more clamps may be used to fix the sheet member 350 to the frame of treatment device 300.

From a procedural standpoint, one additional difference may exist between the delivery and deployment of treatment device 300 compared to treatment device 200. As described above, during delivery, treatment device 200 may be collapsed or otherwise forced into a delivery condition in which the entire treatment device 200 is positioned along a straight line to minimize the delivery profile of the treatment device 200. Although that same process may be possible for treatment device 300, the material forming sheet member 350 would need to either be stretchable (e.g. if formed of certain synthetic materials) or otherwise the coupling of the sides of the sheet member 350 to the first leg 220 and second leg 230 would need to allow for relative movement, e.g. via sliding stitches. Otherwise, if the frame of the treatment device 300 were collapsed to be a single substantially straight line, the sheet member 350 would be in danger of tearing or otherwise being damaged from tension being placed on the sheet member 350. This tearing or damage may be avoided, however, by collapsing the frame of the treatment device 300 for delivery in the manner shown in FIG. 4B. In other words, when in the collapsed condition or the delivery condition, the terminal ends of the frame of the treatment device 300 (e.g. the first curved portion 224 and the second curved portion 234) would fold to point toward one another, instead of pointing away from one another, so that the frame of the treatment device 300 has about double thickness in the folded over areas. Although this process would increase the profile of the treatment device 300 for delivery compared to treatment device 200, the sheet member 350 (not illustrated in FIG. 4B) would be protected from tearing or other damage during delivery by using this method. However, in some embodiments, this increased profile may be desirable. For example, the increased profile may have more of an oval shape in cross-section, compared to the circular shape shown and described in connection with FIG. 3C. For example, the treatment device 300 may be used with a delivery catheter that has a generally oval profile that matches the oval profile of the end portions of the treatment device 300 when in the delivery condition. The matching oval profile may provide an additional level of steering control, since the treatment device 300 may be generally prevented from rotating relative to the delivery catheter while housed by the delivery catheter due to the matching oval profiles.

As with treatment device 200, treatment device 300 may be provided in a plurality of different sizes to better fit within (and provide the desired tension to the native leaflets of) a particular patient's heart valve that is being treated. However, treatment device 300 may also be provided in a plurality of different thicknesses of sheet member 350. For example, if an examination of the patient shows that a gap of 3 mm exists between the native leaflets 136, 138, a treatment device 300 may be provided with a sheet member 350 having a thickness of 5 mm to fill the gap. In some embodiments, the thickness of the sheet member 350 may be tailored to be the same as any native coaptation gap, or slightly larger (or even in some cases slightly smaller) than a native coaptation gap. The coaptation gap may be determined prior to treatment using any suitable imaging modality, such as echocardiography, etc.

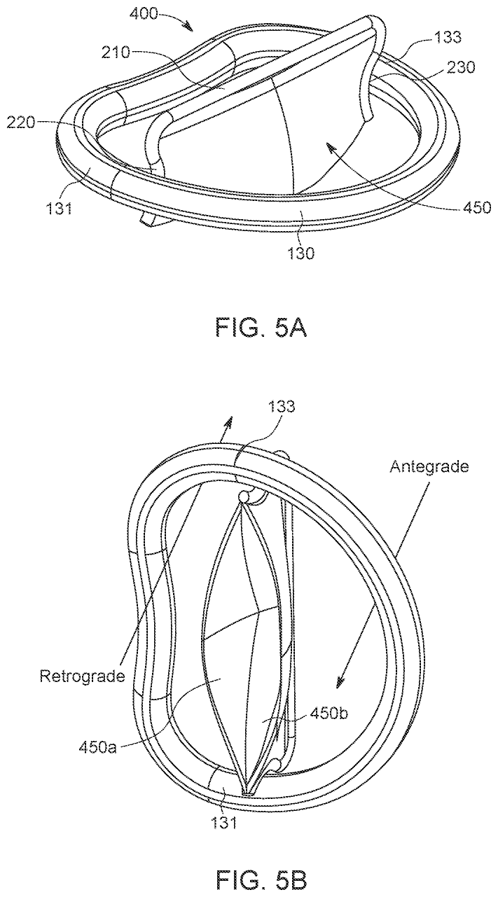

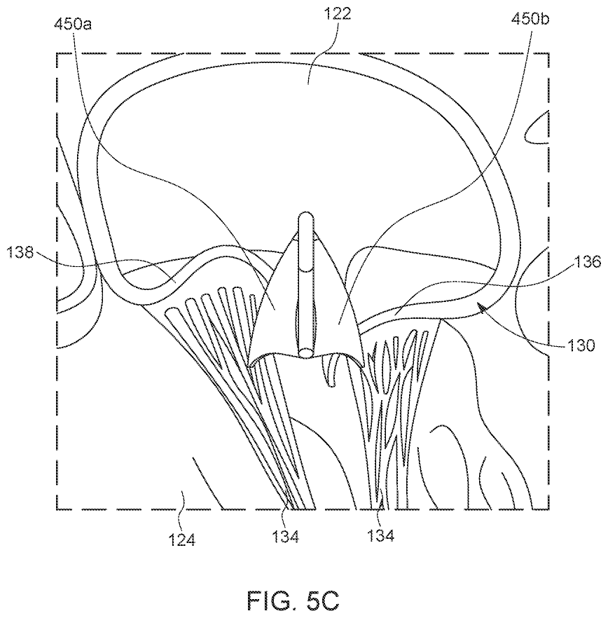

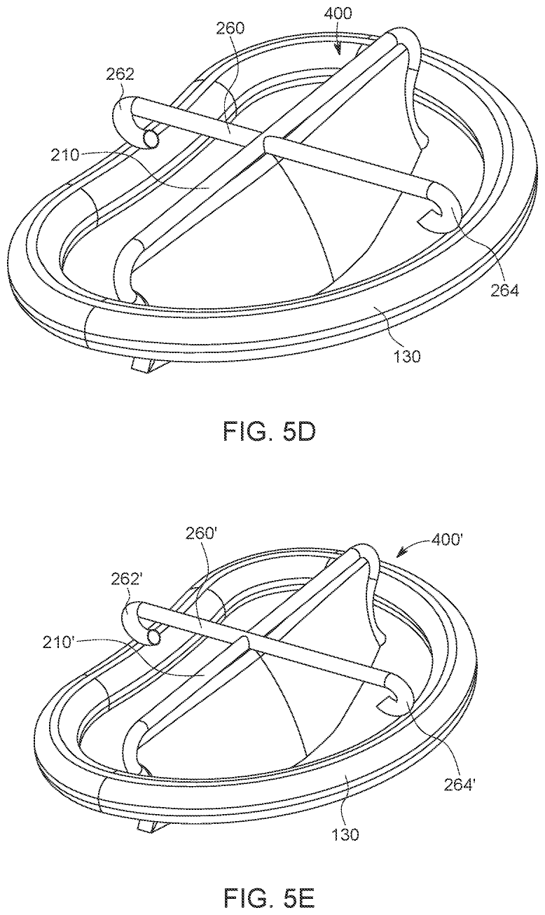

FIGS. 5A-C illustrate a valve treatment device 400 according to another aspect of the disclosure. Treatment device 400 may, effectively, be the treatment device 300 described above, with a modified sheet member 450, where sheet member 450 includes two individual sheets or flaps 450a, 450b. The sheet member 450 may provide a surface against which native valve leaflets 136, 138 may press when the native valve leaflets coapt, but may also tend to expand or billow open upon blood flow (or pressure) in the retrograde direction. By billowing outwardly as the native leaflets 136, 138 press inwardly, the sheet member 450 may help fill any gaps that would otherwise be present between the coapted native valve leaflets 136, 138 in the absence of the sheet member 450.

As with treatment device 300, treatment device 400 may include a frame that is similar or identical to treatment device 200, and thus the frame of the treatment device 400 is not described in greater detail here. The transitioning of treatment device 400 to the collapsed or delivery condition, as well as the actual method for delivering treatment device 400 to the native heart valve, may be similar or identical to that described in connection with treatment device 300, and is thus not described in greater detail here.

The sheet member 450 may be formed of any of the materials described in connection with sheet member 350, and may be attached to the frame in substantially the same way. The main structural difference between sheet member 450 and sheet member 350 is that, while sheet member 350 may be a single piece, sheet member 450 is formed of two separate sheets or flaps 450a, 450b. However, as with sheet member 350, sheet member 450 may be a single sheet that is folded over the central bar 210, but the free ends of the sheet member 450 may not be fixed to one another as may be the case for sheet member 350. Each flap 450a, 450b may be similar or identical to sheet member 350, and the individual flaps 450a, 450b are thus not described in further detail here. The flaps 450a, 450b may be coupled to the frame of the treatment device 400 in substantially the same way as described in connection with sheet member 350. However, it should be understood that the bottom of each flap 450a, 450b, which refers to the portion farthest from central bar 210, is not coupled to one another. With this configuration, a substantially closed volume is formed between the inner surfaces of the two flaps 450a, 450b, with the only pathway into that closed volume being through the area defined between the bottoms of the two flaps 450a, 450b.

After implantation of the treatment device 400, the open area between the bottoms of the two flaps 450a, 450b faces in the outflow direction toward the left ventricle, which is generally toward the bottom of the page in the views of FIGS. 5A and 5C. As the left atrium 122 contracts, the native leaflets 136, 138 open and blood flows through the mitral valve into the left ventricle 124. This blood flow in the antegrade direction may tend to push flaps 450a, 450b together, and significant clearance remains between the outside of the flaps 450a, 450b and the native valve annulus so that the treatment device 400 does not impede blood passing from the left atrium 122 to the left ventricle 124. When the left ventricle 124 contracts to force blood into the aorta, the native leaflets 136, 138 of the mitral valve 130 close (or attempt to close) to prevent blood from flowing in the retrograde direction, which is generally toward the top of the page in the view of FIGS. 5A and 5C. FIG. 5B illustrates the treatment device 400 within the mitral valve 130 from the left ventricle as the left ventricle contracts. The native valve leaflets 136, 138 are omitted for clarity from the view of FIG. 5B, but are illustrated in FIG. 5C. As the left ventricle 124 contracts and retrograde blood flow creates pressure to close the native valve leaflets 136, 138, the retrograde blood flow passes through the open area between the bottoms of the flaps 450a, 450b and into the otherwise closed volume between the two flaps 450a, 450b. As the retrograde blood flow passes into the volume between the flaps 450a, 450b, as best shown in FIG. 5B, the flaps will tend to billow outwardly and provide contact surfaces against which the native leaflets 136, 138 may close to enhance the seal at the mitral valve 130. Although treatment devices 400 and 300 may both be helpful in patients with regurgitation at the mitral valve 130, treatment device 400 may be particularly helpful in patients that have a large gap between the native leaflets 136, 138 when the native valve closes, as the flaps 450a, 450b may be able to fill more space than the single sheet member 350.

Although treatment device 400 may be able to remain fixed in position without additional support, it should be understood that, compared to treatment devices 200 and 300, treatment device 400 would likely experience more forces during normal operation. In particular, during left ventricular contraction, the retrograde blood flow into the volume between the flaps 450a, 450b may be significant and tend to cause the treatment device 400 to want to migrate in the retrograde direction in the left atrium, or to otherwise shift in the anterior or posterior direction (which is generally perpendicular to the direction along which central bar 210 extends). Although the frame of the treatment device 400 may provide sufficient support alone, in other embodiments, additional support features may be provided.

FIG. 5D illustrates treatment device 400 deployed in the native mitral valve 130 with an additional support member 460. The treatment device 400 shown in FIG. 5D is identical to that shown in FIGS. 5A-C, with the exception that the additional support member 460 is provided, and features may be included within central bar 210 to couple the central bar to the additional support feature. Additional support feature 260 may take the form of another bar, for example a tube of shape memory material, such as a nickel titanium alloy like nitinol. When in the deployed condition shown in FIG. 5D, the additional support feature 260 includes a central bar extending generally perpendicularly to central bar 210. In other words, while central bar 210 extends in the medial-lateral direction to contact the native commissures 131, 133, the central bar of the additional support feature 260 extends in the anterior-posterior direction to provide support against anterior-posterior canting or tilting of the treatment device 400. The terminal ends of the additional support feature 260 may include a first curved section 262 and a second curved section 264, with the curved sections being generally U-shaped or generally C-shaped, although other shapes may be suitable. In the illustrated embodiment, when treatment device 400 is deployed in the native mitral valve 130, the first curved section 262 and second curved section 264 curve in the outflow direction (toward the left ventricle 124) and then back in the direction toward sheet member 450. The additional support feature 260, and particularly the first and second curved sections 262, 264, may contact the native valve annulus and/or tissue on the atrial side of the mitral valve 130 to help provide additional positional support to the treatment device 400.

As with the remainder of the frame of treatment device 400, additional support feature 260 is preferably shape set, for example via heat setting, to the shape shown in FIG. 5D, or a similar shape. With this configuration, during delivery within a catheter or other delivery device, the additional support member 260 can be straightened so that it is substantially completely straight, for example being generally cylindrical, to have a small profile. Although when deployed the central bar 210 of the frame of the treatment device 400 is generally perpendicular to the additional support feature 260, the components may be moveable so that, during delivery, the frame of the treatment device 400 and the additional support member 260 both extend along a straight line and are parallel (or almost parallel) with each other to form a small profile for delivery.

There are various ways in which central bar 210 and additional support feature 260 may be formed to be movable relative to one another to allow for small profile delivery is by forming an aperture in the central bar 210, as shown in FIG. 5D, and passing the additional support member 260 through that central aperture to couple the two members so that they are movable, effectively with hinge-like movement. Alternatively, as shown in FIG. 5E, treatment device 400′ may include a central bar 210′, and an aperture may be formed in the additional support member 260′ and the central bar 210′ may be passed through that aperture. The first and second curved sections 262′, 264′ may be similar or identical to those shown and described in connection with FIG. 5D. Other coupling mechanisms may be suitable to allow for the desired functionality. For example, the central bar 210 (or 210′) and additional support member 260 (or 260′) may be sutured or otherwise tied together, and instead of apertures, the bars may include recesses to allow for nesting of the central bar and the additional support member. In other embodiments, the central bar 210 (or 210′) and additional support member 260 (or 260′) may be provided without a tight connection in the delivery catheter during delivery. The central bar 210 (or 210′) and additional support member 260 (or 260′) may be loosely coupled by a suture or wire, so that the treatment device 400 (or 400′) may be delivered first while the additional support member 260 (or 260′) remains in the catheter. The coupling suture may act as a rail so that the additional support member 260 (or 260′) may be delivered second and then drawn into position (e.g. by pulling the suture until a nesting feature, such as a recess, couples the additional support member to the central bar).

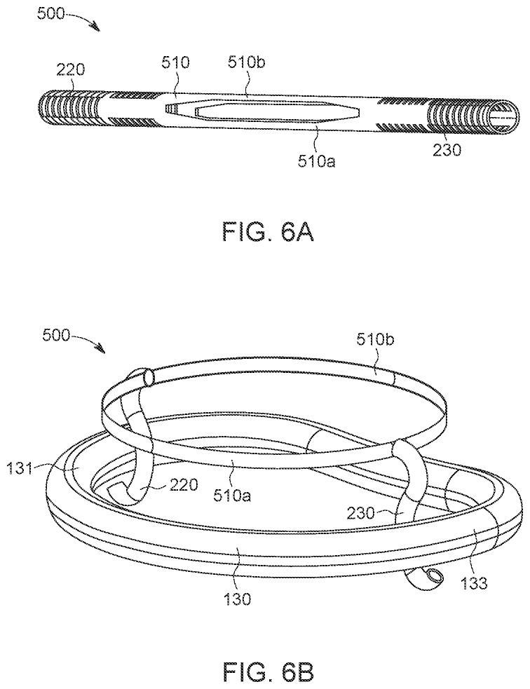

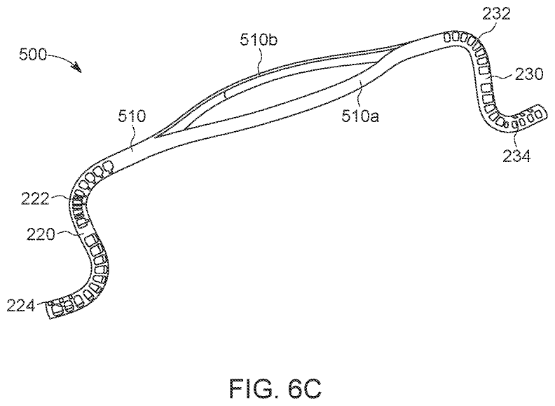

FIG. 6A illustrates a treatment device 500, in a collapsed or delivery configuration, according to another embodiment of the disclosure. Treatment device 500 may be substantially identical to treatment device 200, except for certain differences described below. Thus, anything not specifically described in connection with treatment device 500 should be understood to be similar or identical to the corresponding features and use of treatment device 200. For example, treatment device 500 may be formed as a solid or hollow tube of shape memory material such as a nickel titanium alloy like nitinol. As with treatment device 200, treatment device 500 may include a central bar 510, a first leg 220, and a second leg 230. First leg 220 and second leg 230 of treatment device 500 may be similar or identical to first leg 220 and second leg 230 of treatment device 200, including the option of being solid or hollow, and the option of including opposing segmented sections. In any of the embodiments described herein, the terminal ends of the frame of the treatment device may include a wrap or bulk of fabric or other atraumatic structures to help prevent any unintentional puncturing of tissue during deployment or during normal operation of the repair device.

The main difference between treatment device 500 and treatment device 200 lies in the difference between central bar 210 and central bar 510. As shown in FIG. 6A, the central bar 510 may be formed into two separate segments 510a, 510b that are spaced from one another by a recess or cavity. The segments 510a, 510b may be continuous and/or integral with the remainder of the treatment device 500, including first leg 220 and second leg 230. For example, the segments 510a, 510b may be formed by subtractive manufacturing, including for example via laser cutting or etching, the recess or cavity shown in FIG. 6A. Each segment 510a, 510b may be referred to as a strut, and each segment may be a wall of the tube from which treatment device 500 is formed.

Treatment device 500 is shown in the collapsed or delivery condition in FIG. 5A, which may be generally the same or identical the collapsed or delivery condition of treatment device 200. In other words, treatment device 500 may be substantially in a straight line or a cylindrical shape in the delivery condition. However, as with the other treatment devices described herein, treatment device 500 is preferably shape-set, for example via heat setting, to tend to take the shape shown in FIG. 6B in the absence of applied forces.

FIG. 6B shows treatment device 500 in a deployed or expanded condition within the mitral valve 130. The first leg 220 and second leg 230 of treatment device 500 may have substantially the same shape as described above in connection with treatment device 200 when in the deployed or expanded condition. However, rather than central bar 210 extending in a relatively straight medial-lateral direction, the struts 510a, 510b of central bar 510 are shape set to splay away from each other in an arcuate shape. As shown in FIG. 6B, the set shape of the struts 510a, 510b may together form a generally circular shape. However, the arc of the struts 510a, 510b may be greater or smaller than that shown in FIG. 6B. For example, FIG. 6C illustrates treatment device 500 with struts 510a, 510b having a smaller arc than is shown in FIG. 6B.

The use of a splayed or segmented central bar 510 that opens to form a recess or cavity between the struts 510a, 510b may have a number of benefits. For example, although central bar 210 would not experience significant forces from blood flowing through the mitral valve 130 in the antegrade direction, central bar 510 may experience even less force from blood flowing through the mitral valve in the antegrade direction because blood would tend to flow through the large center opening, as opposed to flowing against the struts 510a, 510b. Another benefit is that the positioning of the struts 510a, 510b when implanted may easily allow for further interventions, and may even support further interventions. For example, if an additional valve repair device, such as one of the leaflet clips mentioned above, needs to be implanted into a patient at a time after the treatment device 500 has been implanted, the treatment device 500 would not serve as an impediment. First, the struts 510a, 510b are positioned away from the center of the mitral valve 130, so any catheters or later devices that need to be passed through the mitral valve would have clearance between the struts. Second, the struts 510a, 510b (like central bar 210) are positioned on the atrial side of the mitral valve 130, so any clips used to clip the native leaflets 136, 138 on the ventricular side of the mitral valve would not be in danger of being disturbed or contacted by the struts 510a, 510b. Further, if a prosthetic mitral valve (particularly a transcatheter prosthetic mitral valve) is to be implanted into native mitral valve 130 at a time after implantation of treatment device 500, the treatment device would not interfere with the prosthetic mitral valve. First, as noted above, the opening between the struts 510a, 510b allows for a catheter to easily pass through, if desired. Second, even if a prosthetic mitral valve, after implantation, extends above the mitral valve annulus 130, the large opening between the struts 510a, 510b allows for clearance between an outer circumference of the prosthetic mitral valve and the interior diameter of the struts 510a, 510b. Third, particularly for expandable prosthetic mitral valves, the struts 510a, 510b may even provide additional anchoring of the prosthetic mitral valve. For example, an expandable prosthetic mitral valve may be deployed so that one exterior portion of the prosthetic mitral valve contacts and is supported by the native mitral valve annulus, while another supra-annular portion of the prosthetic mitral valve contacts and is supported by the struts 510a, 510b. In other word, the struts 510a, 510b may serve as a second anchoring ring, with the native mitral valve annulus being the first anchoring ring, for a later-implanted prosthetic mitral valve.

For all of the embodiments described herein, certain other features may be provided for additional functionality. For example, as noted above, hooks, tines, or any suitable friction enhancing or piercing members may be provided on the legs of the treatment devices to provide for enhanced anchoring of the treatment device upon implantation. In some embodiments, fabrics or other materials may be provided on one or both legs of the treatment device in order to assist with or enhance tissue ingrowth into the treatment device over time. Such materials may be any biocompatible material, including synthetic fabrics such as PTE, PTFE, etc.

As noted above, a procedure in which any of the treatment devices described above is implanted into a native heart valve annulus may be accompanied by other treatment procedures, and the treatment devices described herein need not interfere with those other treatment procedures. For example, a leaflet clip, such as the MitraClip or TriClip device offered by Abbott Labs, may be implanted prior to or after the treatment devices described herein, in a fully separate medical procedure, without interference between the two devices. In some circumstances, any of the treatment devices described herein may be implanted into a patient in the same medical procedure as another device, such as a leaflet clip.

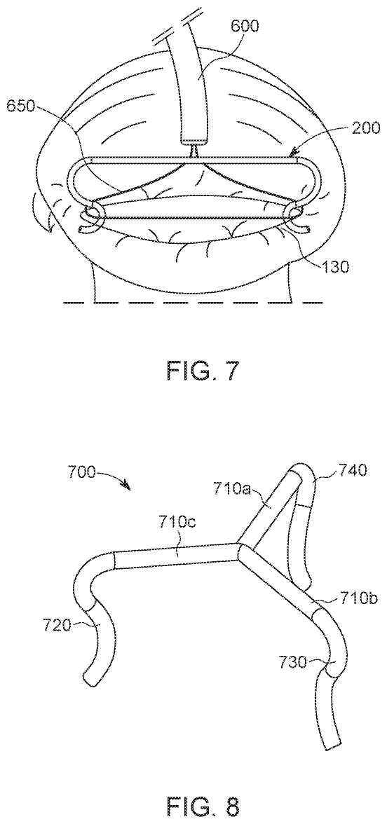

FIG. 7 illustrates treatment device 200 being delivered using a catheter 600 with a tensioning mechanism 650 operably coupled to the treatment device 200 for assisting with placement and/or repositioning of the treatment device 200. For example, a suture or other wire 650 may pass through the catheter 600 so that both free ends of the suture 650 extend proximally through the catheter 600 toward the operator, with a distal loop of the suture 650 coupled to the treatment device. For example, suture 650 may be looped around the second curved portions 224, 234 of the first and second legs 220, 230. Placing tension on the suture 650, for example by pulling the free ends proximally, will tend to shorten the distal loop and pull the legs 220, 230 toward each other, shortening the width between the two second curved portions 224, 234 of the legs. This tensioning mechanism 650 may be used during initial deployment of the treatment device 200 to help control the expansion or one or both legs 220, 230 and/or to help control the placement and positioning of the legs 220, 230 relative to the native commissures. The tensioning mechanism 650 may alternately or additionally be used to assist with repositioning after initial deployment. For example, after initial deployment of the treatment device 200 into the mitral valve 130, if it is determined that repositioning is desirable, the suture 650 may be pulled to draw the legs 220, 230 toward each other to provide clearance for repositioning the treatment device. During controlled delivery of treatment device 200, the tip of the catheter 600 may be drawn up against central bar 210 when the tensioning mechanism 650 (e.g. sutures) are pulled. With this configuration, once tension is applied, the proximal/distal position of the treatment device 200 should be controllable by simply moving the catheter 600 distally or proximally (e.g. distally toward the ventricle or proximally toward the atrium). Additional connection members (e.g. sutures) may be coupled to only one of the legs to provide for additional tilt control. For example, the suture coupled to one leg may be pulled to tilt the device in one rotational direction, or the suture coupled to the other leg may be pulled to tilt the device in the opposite rotational direction.

While FIG. 7 illustrates a tensioning mechanism 650 in the form of a suture that wraps around the treatment device 200, if the treatment device is hollow, the suture (or other wire) may instead be routed through the interior of the tube forming the treatment device 200. For example, suture 650 could instead have a central portion somewhere near the center of central bar 210, with the two ends of the suture passing through opposite legs 220, 230 and then proximally through the catheter 600, such that pulling the suture proximally has the same effect of drawing the legs 220, 230 toward each other.

Whether the tensioning mechanism is on an exterior or through the interior of the treatment device 200, once the operator is satisfied with the final positioning of the treatment device, one free end of the suture 650 may be pulled proximally, until the other end passes distally through the catheter, disconnects from the treatment device, and then passes proximally back through the catheter 600 to full disconnect the suture 650 from the treatment device 200. Although the tensioning mechanism 650 is only described in connection with treatment device 200, it should be understood that it may be used with any of the treatment devices described herein.

The treatment devices described above have generally been described in connection with treatment of a bi-leaflet valve, such as the native mitral valve 130. However, it should be understood that the concepts described in connection with the treatment devices disclosed above may also be applied to tri-leaflet valves, such as the tricuspid valve, the aortic valve, or the pulmonary valve. For example, FIG. 8 is a perspective view of a treatment device 700 configured for use in tri-leaflet native valve. Treatment device 700 may have two main structural differences compared to treatment deice 200. First, treatment device 700 includes three arms 720, 730, 740 instead of two, with each arm being substantially similar or identical to arms 220, 230. Second, while treatment device 200 includes a single continuous central bar 210, treatment device 700 includes three central bars 710a-c that meet in a center and extend at about 120 degrees apart from one another to the respective three legs 720, 730, 740. As with treatment device 200, each leg 720, 730, 740 of treatment device 700 may include a first curved portion with a convex outer surface and a second curved portion with a concave outer surface, the concave outer surfaces configured to nest within the corresponding three native commissures of the native tri-leaflet valve being treated. Treatment device 700 may be formed as solid tube members, or hollow tube members, with or without segmented portions similar to those described in connection with treatment device 200. All of the variations described in connection with treatment device 200 may apply to treatment device 700 as well. For example, fabric for ingrowth and/or enhanced texturization or anchoring features may be provided on the legs 720, 730, 740 of treatment device 700. A sheet of tissue or fabric in a tri-lobe or tri-cusp configuration may be provided with treatment device 700, with one sheet portion extending from each of the central bars 710a-c and/or along each leg 720, 730, 740, with the sheet being configured to simply fill any gaps between the leaflets of the native tri-leaflet valve, similar to treatment device 300. Still further, instead of single sheet portions extending along each central bar 710a-c, one sheet member may extend along each adjacent pair of central arms 710a-c, so that a volume is created between adjacent pairs of sheet members, similar to treatment device 400. Still further, each central bar 710a-c may be formed with an opening or as a split bar, with the end of each bar near its associated leg being closed, but with the openings of each bar meeting at the center where three central bars meet. In this embodiment, when the treatment device 700 is deployed, it may change shape so that the central bars in combination form a generally circular shape that is open, similar to treatment device 500.

From a procedural standpoint, one of the main differences between a tri-leaflet treatment device similar to treatment device 700 compared to a bi-leaflet treatment device similar to treatment devices 200-500 is the size of the device in the collapsed condition. The bi-leaflet treatment devices described above may be collapsed into a single straight line or cylindrical tube (such as with treatment devices 200, 500) or with only portions of the legs folding to form a double profile area (such as with treatment devices 300, 400). Treatment device 700, when collapsed and loaded into a delivery device in the delivery condition, may have two any two central bars (and their associated legs) pointing in one direction, with the remaining central bar (and its associated leg) pointing in the opposite direction, so that about half the length of the treatment device 700 is “doubled up” when in the delivery condition. If sheet members are provided with treatment device 700, portions of the legs 720, 730, 740 may be folded for delivery, similar to the description in connection with FIG. 4B.

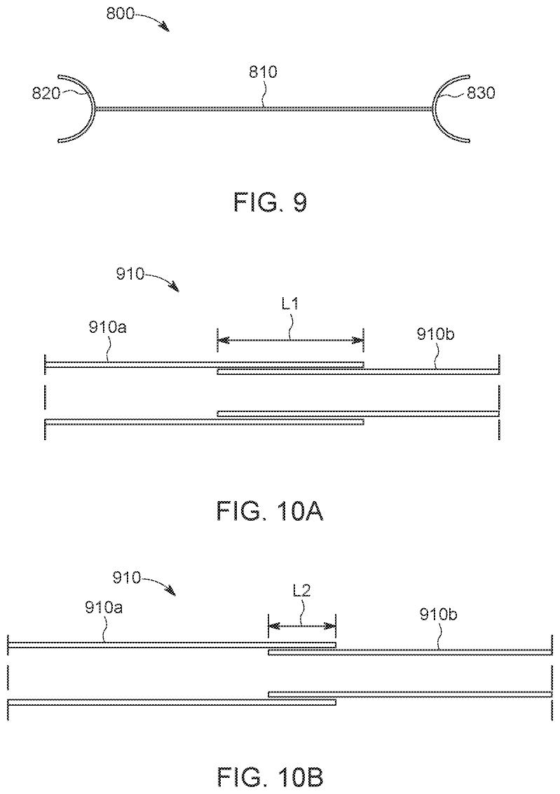

FIG. 9 shows another treatment device 800 for a bi-leaflet valve. Although shown in generally simplified form, it should be understood that treatment device 800 may be formed of a shape memory material, for example a solid, hollow, or braided tube, which may have a cross-sectional profile of circular, oval, rectangular, etc. As with treatment device 200, treatment device 800 may have a central bar 810. However, the terminal ends of the central bar 810 may transition directly into two ends 820, 830 that each are generally “C”- or “U”-shaped in the absence of applied forces. Although the two ends 820, 830 may be integral with the central bar 810, in other embodiments, the two ends 820, 830 may be formed separately and then coupled to central bar 810. In use, the two ends 820, 830 may be collapsed so that the two terminal portions of each of the two ends 820, 830 faces away from the central bar, and then loaded into a delivery device. Upon deployment, the first end 820 may be deployed to engage one of the native commissures, and the second end 830 may be deployed to engage the other one of the native commissures. In other words, each of the two ends 820, 830 may engage the respective commissures by having one portion extending superior to the annulus, and one portion extending inferior to the annulus, so that the native commissures nest within the “C”-shape of the ends 820, 830. In the shape set condition shown in FIG. 9, the central bar 810 may be generally straight extending between the apices of the “C”-shapes of the two ends 820, 830. With this configuration, when the treatment device 800 is deployed within the native valve annulus, the central bar 810 extends substantially within the plane of the native valve annulus. The central bar 810 may provide additional structure against which the native leaflets may press when the native leaflets transition to the co-apted condition.

FIGS. 10A-B illustrate a central bar 910 that may be used with suitable ones of the treatment devices described above, for example treatment device 200. Instead of being formed as a single integral member, central bar 910 is formed from multiple pieces that may translate relative to one another to increase or decrease the length between opposite ends of the treatment device, for example to increase or decrease the amount of force applied to the native valve upon deployment. In the illustrated embodiment, central bar 910 includes a first bar portion 910a having a larger diameter than bar portion 910b, so that bar portions 910a-b may couple together in a telescoping manner. In other words, in a first shorter length condition, shown in FIG. 10A, there is a first relatively large length of overlap L1 between the two bar portions 910a-b. In a second larger length condition, shown in FIG. 10B, there is a second relatively small length of overlap L2 between the two bar portions 910a-b. Any suitable mechanism may be used to actuate the central bar 910 to change the effective length of the central bar. For example, sliding mechanisms may be used, such as a ratchet-and-pawl mechanism to allow for one-way sliding of the first bar portion 910a relative to the second bar portion 910b. In other embodiments, rotating mechanisms may be used, for example by having a threaded connection between the two bar portions 910a, 910b. In other embodiments, the central bar 910 may have three pieces in a turnbuckle configuration, with two substantially equally sized central bar portions that are coupled to a turnbuckle mechanism positioned between the two central bar portions. As should be understood from the above, the effective length of the treatment device that incorporates central bar 910 may be fine-tuned before, or preferably after, deployment into the native valve annulus to adjust the level of force applied to the native valve annulus.

All of the treatment devices described herein are highly minimally invasive compared to current known leaflet repair devices and prosthetic heart valve replacement devices. Further, even after implantation of any of the treatment devices described herein, additional interventions at a later time will generally still be possible, and in some embodiments, the treatment device may even provide assistance for those later interventions, such as treatment device 500 having struts 510a, 510b that may provide additional anchoring points for a later-implanted prosthetic heart valve. Still further, some patients may not be able to accept any treatment devices that extend any appreciable distance into a chamber of the heart. For example, in patients with left ventricular cavity obliteration (“LVCO”), the left ventricle contracts to such a point that any external structure within the left ventricle (e.g. a portion of a prosthetic mitral valve extending any distance into the left ventricle) would occlude the left ventricular outflow tract (“LVOT”) during ventricular systole. In those patients, treatment devices that extend any appreciable distance into the left ventricle may not be viable treatment options. The treatment devices described herein, however, do not extend any appreciable distance in the outflow direction beyond the native valve being treated. Thus, the treatment devices described herein would be suitable for use even in patients with LVCO.

According to one aspect of the disclosure, a medical device for treating a bi-leaflet heart valve comprises:
a frame formed of shape-memory material, the frame including a central bar extending between a first leg and a second leg of the frame, the frame being transitionable between a delivery condition and a deployed condition, the frame being formed of a solid or hollow tube,
wherein the first leg and the second leg each having a curved portion with a concave outer surface in the deployed condition, the concave outer surface sized and shaped to engage a corresponding native commissure of the bi-leaflet heart valve so that, when the frame is deployed within the bi-leaflet heart valve, the central bar bridges across the bi-leaflet heart valve; and/or
the shape-memory material is a nickel titanium alloy; and/or
in the delivery condition, the central bar, the first leg, and the second leg are substantially coaxial; and/or
the curved portions of the first leg and the second leg are each first curved portions, and the first leg and the second leg each include a second curved portion with convex outer surface, the second curved portion of connecting the first curved portion to the central bar; and/or
the first curved portions of the first leg and the second leg each include a first spine and a group of first cut-outs, and the second curved portions of the first leg and the second leg each include a second spine and a group of second cut-outs; and/or
the first spines are each positioned diametrically opposed to the corresponding groups of first cut-outs, and the second spines are each positioned diametrically opposed to the corresponding groups of second cut-outs; and/or
the first spine of the first leg is positioned diametrically opposed to the second spine of the first leg, and the first spine of the second leg is positioned diametrically opposed to the second spine of the second leg; and/or
the central bar is formed by a first strut and a second strut with an opening therebetween; and/or
in the deployed condition, the first strut and the second strut are each arcuate so that a generally circular or elliptical space is formed between the first strut and the second strut; and/or
in the delivery condition, the central bar extends along a first longitudinal axis, and the first and second legs each extend along longitudinal axes that are offset from the first longitudinal axis; and/or
a single sheet member, and in the deployed condition of the frame, the single sheet member is bounded on three sides by the central bar, the first leg, and the second leg, and unbounded by the frame on a fourth side; and/or
the single sheet member is a substantially flat tissue sheet; and/or
two sheet members, and in the deployed condition of the frame, each of the two sheet members are bounded on three sides by the central bar, the first leg, and the second leg, and unbounded by the frame on a fourth side; and/or
a volume is defined between the two sheet members, the volume being accessible via space between the fourth side of each of the two sheet members in the deployed condition of the frame; and/or
the frame includes an additional support bar, and in the deployed condition of the frame, the additional support bar extends substantially perpendicular to the central bar; and/or
the additional support bar is movably coupled to the central bar so that the additional support bar may rotate toward or away from the central bar between the delivery condition and the deployed condition; and/or
the medical device is part of a heart valve repair system that further includes a catheter configured to receive the medical device in the delivery condition; and a tensioning mechanism configured to be positioned within the catheter and be operably coupled to the medical device so that, in the deployed condition of the medical device, proximal movement of the tensioning mechanism causes the curved portions of the first leg and the second leg to move closer to one another; and/or
the tensioning mechanism is a wire or a suture; and/or
in the deployed condition of the medical device, the wire or suture loops around the concave outer surfaces of the curved portions of the first leg and the second leg; and/or
in the deployed condition of the medical device, the wire or suture passes through a hollow interior of the medical device and exits the medical device through terminal ends of the curved portions of the first leg and the second leg.

According to another aspect of the disclosure, a method of treating a mitral valve of a patient comprises:
loading a medical device into a catheter in a delivery condition;
advancing the catheter through a vasculature of the patient to a right atrium of the patient, through an atrial septum, and into a left atrium of the patient while the catheter maintains the medical device in the delivery condition; and
deploying the medical device from the catheter into engagement with the mitral valve, the medical device transitioning from the delivery condition to a deployed condition during the deployment;
wherein when the medical device is in the deployed condition, a first leg of the medical device engages a first commissure of the mitral valve, a second leg of the medical device engages a second commissure of the mitral valve, and a central bar extends from the first leg to the second leg so that the medical device presses outwardly on the first commissure and the second commissure.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A medical device for treating a bi-leaflet heart valve, the medical comprising:
a frame formed of shape-memory material, the frame including a central bar extending between a first leg and a second leg of the frame, the frame being transitionable between a delivery condition and a deployed condition,
wherein the first leg and the second leg each having a curved portion with a concave outer surface in the deployed condition, the concave outer surface sized and shaped to engage a corresponding native commissure of the bi-leaflet heart valve so that, when the frame is deployed within the bi-leaflet heart valve, the central bar bridges across the bi-leaflet heart valve.

2. The medical device of claim 1, wherein the shape-memory material is a nickel titanium alloy.

3. The medical device of claim 1, wherein in the delivery condition, the central bar, the first leg, and the second leg are substantially coaxial.

4. The medical device of claim 3, wherein the curved portions of the first leg and the second leg are each first curved portions, and the first leg and the second leg each include a second curved portion with convex outer surface, the second curved portion of connecting the first curved portion to the central bar.

5. The medical device of claim 4, wherein the first curved portions of the first leg and the second leg each include a first spine and a group of first cut-outs, and the second curved portions of the first leg and the second leg each include a second spine and a group of second cut-outs.

6. The medical device of claim 5, wherein the first spines are each positioned diametrically opposed to the corresponding groups of first cut-outs, and the second spines are each positioned diametrically opposed to the corresponding groups of second cut-outs.

7. The medical device of claim 6, wherein the first spine of the first leg is positioned diametrically opposed to the second spine of the first leg, and the first spine of the second leg is positioned diametrically opposed to the second spine of the second leg.

8. The medical device of claim 3, wherein the central bar is formed by a first strut and a second strut with an opening therebetween.

9. The medical device of claim 8, wherein in the deployed condition, the first strut and the second strut are each arcuate so that a generally circular or elliptical space is formed between the first strut and the second strut.

10. The medical device of claim 1, wherein in the delivery condition, the central bar extends along a first longitudinal axis, and the first and second legs each extend along longitudinal axes that are offset from the first longitudinal axis.

11. The medical device of claim 10, further comprising a single sheet member, and in the deployed condition of the frame, the single sheet member is bounded on three sides by the central bar, the first leg, and the second leg, and unbounded by the frame on a fourth side.

12. The medical device of claim 11, wherein the single sheet member is a substantially flat tissue sheet.

13. The medical device of claim 10, further comprising two sheet members, and in the deployed condition of the frame, each of the two sheet members are bounded on three sides by the central bar, the first leg, and the second leg, and unbounded by the frame on a fourth side.

14. The medical device of claim 13, wherein a volume is defined between the two sheet members, the volume being accessible via space between the fourth side of each of the two sheet members in the deployed condition of the frame.

15. The medical device of claim 14, wherein the frame includes an additional support bar, and in the deployed condition of the frame, the additional support bar extends substantially perpendicular to the central bar.

16. The medical device of claim 15, wherein the additional support bar is movably coupled to the central bar so that the additional support bar may rotate toward or away from the central bar between the delivery condition and the deployed condition.

17. A heart valve repair system, comprising:
the medical device of claim 1;
a catheter configured to receive the medical device in the delivery condition; and
a tensioning mechanism configured to be positioned within the catheter and be operably coupled to the medical device so that, in the deployed condition of the medical device, proximal movement of the tensioning mechanism causes the curved portions of the first leg and the second leg to move closer to one another.

18. The heart valve repair system of claim 17, wherein the tensioning mechanism is a wire or a suture.

19. The heart valve repair system of claim 18, wherein in the deployed condition of the medical device, the wire or suture loops around the concave outer surfaces of the curved portions of the first leg and the second leg.

20. The heart valve repair system of claim 18, wherein in the deployed condition of the medical device, the wire or suture passes through a hollow interior of the medical device and exits the medical device through terminal ends of the curved portions of the first leg and the second leg.

21. A method of treating a mitral valve of a patient, the method comprising:
loading a medical device into a catheter in a delivery condition;
advancing the catheter through a vasculature of the patient to a right atrium of the patient, through an atrial septum, and into a left atrium of the patient while the catheter maintains the medical device in the delivery condition; and
deploying the medical device from the catheter into engagement with the mitral valve, the medical device transitioning from the delivery condition to a deployed condition during the deployment;
wherein when the medical device is in the deployed condition, a first leg of the medical device engages a first commissure of the mitral valve, a second leg of the medical device engages a second commissure of the mitral valve, and a central bar extends from the first leg to the second leg so that the medical device presses outwardly on the first commissure and the second commissure.
